(19) United States
(12) Patent Application Publication
(10) Pub. No.: US 2025/0303127 A1
(43) Pub. Date: Oct. 2, 2025

(54) MULTI-MODAL FLUID MANAGEMENT MODULE

(71) Applicant: NEUFLUENT LLC, Seattle, WA (US)

(72) Inventors: Samuel Robert Browd, Mercer Island, WA (US); Ryan J. Douglas, Seattle, WA (US)

(73) Assignee: NEUFLUENT LLC, Seattle, WA (US)

(21) Appl. No.: 19/093,734

(22) Filed: Mar. 28, 2025

Related U.S. Application Data
(60) Provisional application No. 63/571,079, filed on Mar. 28, 2024.

(51) Int. Cl. A61M 27/00; A61N 5/06

(52) U.S. Cl. CPC A61M 27/006; A61N 5/0601; A61N 5/062; A61M 2205/051; A61M 2205/18; A61M 2205/3334; A61M 2205/3344; A61M 2230/10; A61N 2005/0612; A61N 2005/0626; A61N 2005/0645; A61N 2005/0663

(57) ABSTRACT

Apparatus and associated methods relate to multi-modal fluid management module (MMFMM) including a conduit in fluid communication with a cerebrospinal fluid (CSF) filled region of the brain, two or more electrodes in electrical contact with a region of a brain or dura, and at least one optical emitter configured to emit light to a target spot within a brain. The light may, for example, include a wavelength suitable for optogenetics. The MMFMM may, for example, include at least one pressure sensor mechanically coupled to the conduit configured to measure intracranial pressure. In an illustrative example, the MMFMM may, for example, include a valve configured to control fluid flow in and out of the conduit. Various embodiments may advantageously provide a system to monitor and control intracranial pressure and monitor and stimulate electrical activity of the brain (e.g., brain activity).

### CROSS-REFERENCE TO RELATED APPLICATIONS

This application is a non-provisional application and claims the benefit of U.S. Application Ser. No. 63/571,079, titled “Smart Cerebral Shunt,” filed by Samuel Robert Browd and Ryan J. Douglas on Mar. 28, 2024.

This application incorporates the entire contents of the foregoing application(s) herein by reference.

The subject matter of this application may have common inventorship with and/or may be related to the subject matter of the following:
U.S. application Ser. No. 19/053,113, titled “Multi-Modal Physiological Sensor,” filed by Samuel Robert Browd, et al., on Feb. 12, 2025.
U.S. Application Ser. No. 63/488,412, titled “Dynamic Shunt Systems,” filed by Samuel Robert Browd, et al., on Mar. 3, 2023.
U.S. Application Ser. No. 63/365,407, titled “Distributed Sensing and Control of Cerebrospinal Fluid,” filed by Samuel Robert Browd, et al., on May 26, 2022.
U.S. Application Ser. No. 63/477,162, titled “Cerebrospinal Fluid Polarization,” filed by Samuel Robert Browd, et al., on Dec. 23, 2022.
U.S. Application Ser. No. 63/477,158, titled “Central Nervous System Monitoring and Intervention,” filed by Samuel Robert Browd, et al., on Dec. 23, 2022.
U.S. Application Ser. No. 63/552,563, titled “Multi-Modal Physiological Sensor,” and filed by Samuel Robert Browd on Feb. 12, 2024.
U.S. Provisional Application Ser. No. 63/552,894, titled “CONTINUOUS NEUROCARE,” filed by Samuel Robert Browd and Ryan J. Douglas on Feb. 13, 2024.

This application incorporates the entire contents of the foregoing application(s) herein by reference.

### TECHNICAL FIELD

Various embodiments relate generally to an implantable physiological device, particularly, for example, to monitor and/or treat neurological conditions.

### BACKGROUND

Neurological diseases encompass a broad spectrum of disorders affecting the brain, spinal cord, and nerves, leading to impairments in movement, cognition, and overall neurological function. These diseases include neurodegenerative conditions such as Alzheimer's and Parkinson's, autoimmune disorders like multiple sclerosis, and acute conditions such as strokes. Given their complexity, neurological diseases often have multifaceted treatment approaches aimed at slowing disease progression, alleviating symptoms, and improving patients' quality of life. An aspect of neurological disease treatment involves regulating cerebrospinal fluid (CSF) pressure and targeting specific brain regions for therapeutic activation. Conditions like hydrocephalus may involve the use of shunts to drain excess CSF and relieve intracranial pressure, preventing further neurological damage. Additionally, advanced neurostimulation techniques are being explored to modulate neural activity and restore function in conditions such as depression, epilepsy, and neurodegenerative disorders. These treatments represent promising frontiers in the ongoing effort to treat and manage neurological diseases effectively. Optogenetics is an emerging technique that offers precise control over neural activity by using light to activate or inhibit specific neurons. This approach involves genetically modifying neurons to express light-sensitive ion channels, such as channelrhodopsins or halorhodopsins, which allow for targeted stimulation or suppression when exposed to specific wavelengths of light. By enabling researchers to manipulate neural circuits with high spatial and temporal resolution, optogenetics may provide potential insights into the mechanisms underlying neurological diseases, including Parkinson's disease, epilepsy, and psychiatric disorders. Additionally, ongoing research aims to translate optogenetic therapies into clinical applications, potentially offering highly specific and minimally invasive treatments for conditions that involve dysfunctional neural activity.

### SUMMARY

Apparatus and associated methods relate to multi-modal fluid management module (MMFMM) including a conduit in fluid communication with a cerebrospinal fluid (CSF) filled region of the brain, two or more electrodes in electrical contact with a region of a brain or dura, and at least one optical emitter configured to emit light to a target spot within a brain. The light may, for example, include a wavelength suitable for optogenetics. The MMFMM may, for example, include at least one pressure sensor mechanically coupled to the conduit configured to measure intracranial pressure. In an illustrative example, the MMFMM may, for example, include a valve configured to control fluid flow in and out of the conduit. Various embodiments may advantageously provide a system to monitor and/or control intracranial pressure and/or monitor and/or modulate electrical activity of the brain (e.g., brain activity).

Various embodiments may achieve one or more advantages. For example, some embodiments may advantageously monitor and/or treat neurological diseases in an ambulatory setting. Some embodiments may, for example, advantageously autonomously control intracranial pressure and CSF pulsatility within a patient's skull. Some implementations may, for example, enable precise and targeted neuromodulation within a patient's brain. Some embodiments may, for example, provide wireless monitoring enabling the ability to track intracranial pressure data and/or brain activity data remotely. Some embodiments may advantageously provide real-time feedback on the effectiveness of neurological treatments, enabling quick adjustments to therapeutic interventions. Some implementations may, for example, advantageously enable continuous monitoring of brain activity and intracranial pressure, reducing the amount of hospital visits and invasive procedures. Some implementations may, for example, advantageously enhance patient safety by automatically detecting and responding to critical changes in brain activity or intracranial pressure.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary multi-modal fluid management module (MMFMM) employed in an illustrative use-case scenario.

FIG. 2 depicts a schematic of another exemplary embodiment of an MMFMM.

FIG. 3 depicts a schematic of another exemplary embodiment of an MMFMM.

FIG. 4 is a block diagram depicting an exemplary multi-modal fluid management module engine (MMFMME).

FIG. 5 is a flowchart illustrating an exemplary method of regulating intracranial pressure including exemplary operations performed by a MMFMME.

FIG. 6 is a flowchart illustrating an exemplary method of modulating brain activity via electrodes including exemplary operations that may be performed by a MMFMME.

FIG. 7 is a flowchart illustrating an exemplary method of modulating brain activity via optogenetics including exemplary operations that may be performed by a MMFMME.

Like reference symbols in the various drawings indicate like elements.

### DETAILED DESCRIPTION OF ILLUSTRATIVE EMBODIMENTS

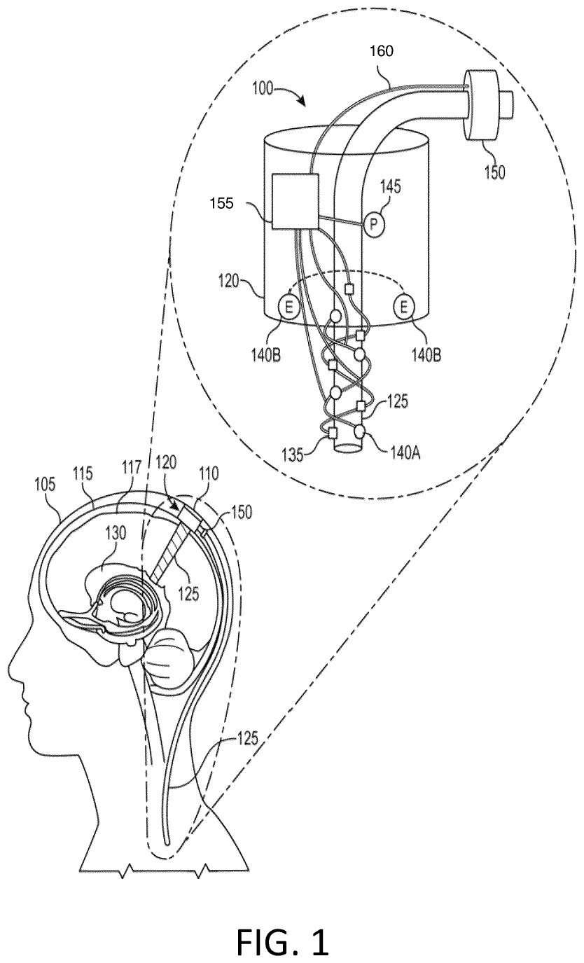

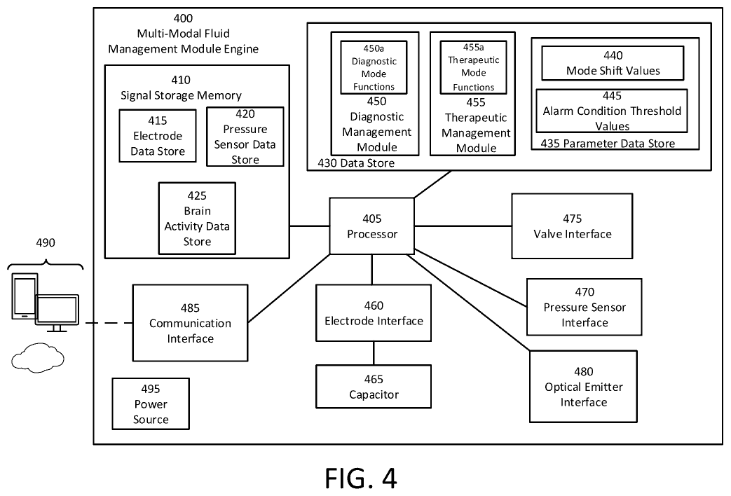

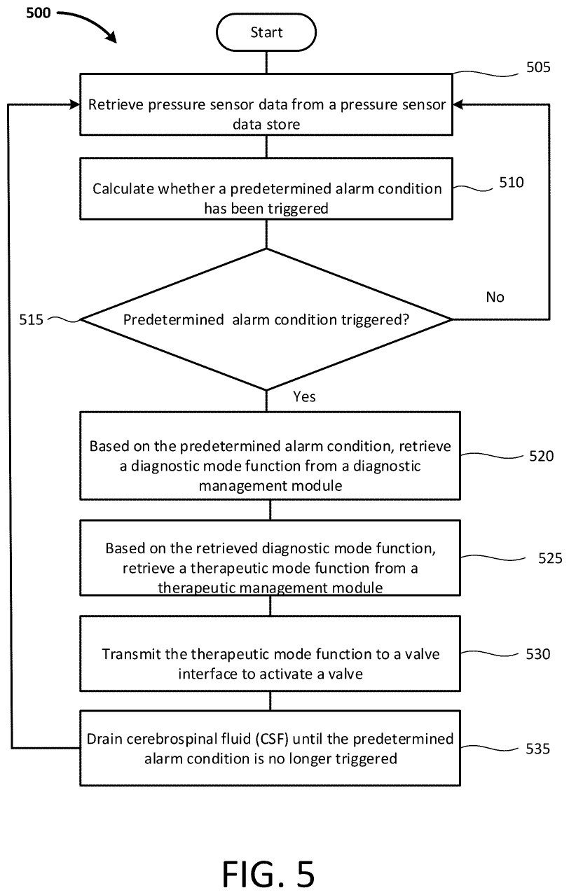

To aid understanding, this document is organized as follows. First, to help introduce discussion of various embodiments, a multi-modal fluid management module (MMFMM) is introduced in an illustrative use-case scenario with reference to FIG. 1. Second, that introduction leads into a description with reference to FIGS. 1-3 of some exemplary embodiments of a MMFMM. Third, with reference to FIG. 4, a block diagram of an exemplary a multi-modal fluid management module engine (MMFMME) is introduced. Fourth, with reference to FIGS. 5-7, exemplary methods of a MMFMM regulating intracranial pressure and modulating electrical activity of the brain (e.g., brain activity) are introduced including exemplary operations performed by an MMFMME. Finally, the document discusses further embodiments, exemplary applications and aspects relating to an MMFMM.

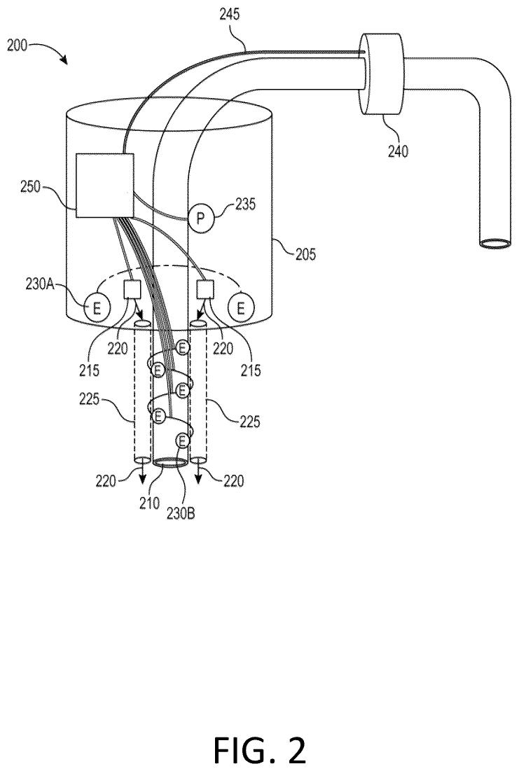

FIG. 1 depicts an exemplary multi-modal fluid management module (MMFMM) 100 employed in an illustrative use-case scenario. The illustrative use-case scenario may, for example, include a surgical scenario where a surgeon implants within a patient's skull 105 the MMFMM 100, for example, to treat a neurological disease. The patient's skull includes an aperture 110 configured to receive the MMFMM 100. In some implementations, for example, the MMFMM 100 may engage with the aperture 110 in a substantially similar arrangement as the MMSMS 120 and the housing 205 described at least with reference to FIGS. 1-2 of U.S. application Ser. No. 19/053,113, titled “Multi-Modal Physiological Sensor,” filed by Samuel Robert Browd, et al., on Feb. 12, 2025.

As will be described in further detail with reference to FIGS. 1-7, the MMFMM 100 may, for example, include electrocochleography (EEG) monitoring and neuromodulation activity as well as cerebrospinal fluid (CSF) pulsatility monitoring and regulation capabilities. Pulsatility, for example, may refer to the variation of CSF pressure and flow due to the cardiac cycle, respiration, and other physiological factors. In various implementations, the installed MMFMM 100 may, for example, enable precise control of neural activity via optogenetics and electrical brain stimulation while configured to regulate intracranial pressure.

A dura 115 and a brain 117 are enclosed within the patient's skull 105. The MMFMM 100 includes a housing 120 in operative contact with the dura 115. The housing 120 encloses a conduit 125. The conduit 125 extends longitudinally out a bottom surface of the housing 120. In some implementations, the conduit 125 may, for example, extend longitudinally out a top surface of the housing 120 and take a radial turn to follow the contour of a subcutaneous surface. In some embodiments, the conduit 125 may, for example, extend outside the radial outer wall of the housing 120 which may advantageously have a lower profile for running the conduit 125 with respect to the subcutaneous surface.

The conduit 125 is disposed in a CSF filled region 130 of the brain 117 via an aperture in the dura 115. This portion of the conduit 125 is positioned within a subdural space. The conduit 125 is configured to be in fluid communication with the CSF filled region 130 such that the conduit 125 drains CSF from the CSF filled region 130. The CSF filled region 130 may, for example, include a ventricle of the brain, as depicted. The conduit 125 extends radially from the housing 120 subcutaneously, as depicted. The conduit 125 may, for example, extend to a remote location. The remote location may, for example include another drainage location in the patient's body. The conduit 125 may, for example, extend radially outside the skull 105 to a remote location external to the patient. The conduit 125 may, for example, advantageously help regulate intracranial pressure. For example, the conduit 125 may advantageously lower intracranial pressure through draining CSF from the CSF filled region 130.

An at least one optical emitter 135 operatively couples the portion of the conduit 125 in the subdural space. The at least one optical emitter 135 may, for example, helically wrap along the outer surface of the conduit 125, as depicted. The at least one optical emitter 135 may, for example electrically connect to each other via a series circuit, as depicted. The at least one optical emitter 135 may include a light emitting diode (LED), for example. The at least one optical emitter 135 is configured to emit light to a target spot within the brain 117 at a wavelength suitable for optogenetics. By way of example, and not limitation the wavelength may include a blue light (˜470 nm). By way of example, and not limitation the wavelength may include yellow/green light (˜530-590 nm). By way of example, and not limitation the wavelength may include red light (˜620-700 nm). The at least one optical emitter 135 may, for example, advantageously modulate electrical activity of the brain 117 (e.g., brain activity) via optogenetics. For example, the brain 117 may include genetically modified neurons that express light-sensitive ion channels such that the at least one optical emitter 135 emits a light suitable to activate the light-sensitive ion channels, thus enabling the depolarization of the genetically modified neurons and triggering action potentials. The at least one optical emitter 135 may, for example, advantageously emit a wide array of light in a substantially omnidirectional pattern directed toward a substantial region of the brain 117 such that the MMFMM 100 may, for example, selectively stimulate neural activation of genetically modified neurons configured for optogenetics at a section of the brain.

The MMFMM 100 includes two or more electrodes 140A and 140B. The two or more electrodes 140A operatively couple the portion of the conduit 125 in the subdural space. For example, the two or more electrodes 140A may helically wraps along the outer surface of the conduit 125 in the subdural space such that the two or more electrodes 140A are in electrical contact with a region of the brain 117. The housing 120 encloses the two or more electrodes 140B such that the two or more electrodes 140B are in electrical contact with a region of the dura 115. The two or more electrodes 140A and 140B may, for example, advantageously provide EEG monitoring capabilities. The two or more electrodes 140A and 140B may advantageously provide electrical brain stimulation capabilities, for example.

Some embodiments of the MMFMM 100 may include features and methods related to regulating CSF pulsatility. CSF pulsatility is the variation in pressure and flow of CSF due to the cardiac cycle, respiratory cycle, or other factors. Regulating CSF pulsatility may have beneficial effects on the treatment of various neurological conditions, such as hydrocephalus, idiopathic intracranial hypertension, or Chiari malformation.

For example, some embodiments of the MMFMM 100 may include at least one pressure sensor 145. The at least one pressure sensor 145 is operably coupled to the conduit 125, as depicted. The at least one pressure sensor 145 may, for example, be arranged in a substantially similar manner to the at least one pressure sensor 150, at least one pressure sensor 230, and at least one pressure sensor 535, as described at least with reference to FIGS. 1-2, and 5 of U.S. application Ser. No. 19/053,113, titled “Multi-Modal Physiological Sensor,” filed by Samuel Robert Browd, et al., on Feb. 12, 2025.

The MMFMM 100 may, for example, include a valve 150 operatively coupled to the conduit 125. The conduit 125 extends radially from the housing 120 to the valve 150. The valve 150 may, for example, advantageously adjust the rate of CSF flow based on the sensed CSF pressure by the at least one pressure sensor 145. For example, the valve 150 may be configured to increase the rate of CSF flow when the pressure exceeds a predetermined threshold and decrease the rate of CSF flow when the pressure falls below the threshold. This may reduce the peak pressure and dampen the pulsatility of CSF.

The MMFMM 100 includes a multi-modal fluid management engine (MMFMME) 155. The MMFMME 155 communicably couples to the at least one optical emitter 135, the two or more electrodes 140A and 140B, the at least one pressure sensor 145, and the valve 150 via connectors 160. The connectors 160 may, for example, include a lead. The connectors 160 may, for example, include a wireless connection. As will be described in more detail with reference to FIGS. 4-7, the MMFMME 155 may, for example, perform operations via the at least one optical emitter 135, the two or more electrodes 140A and 140B, the at least one pressure sensor 145, and the valve 150. The MMFMM 100 may, for example, advantageously be employed in an ambulatory setting.

FIG. 2 depicts a schematic of another exemplary embodiment of an MMFMM 200. The MMFMM 200 includes a housing 205. The housing 205 encloses a conduit 210. The conduit 210 extends in a substantially similar arrangement to the conduit 125. The housing 205 encloses an at least one optical emitter 215. The at least one optical emitter 215 emits a light 220. The light 220 may, for example, include a light at a wavelength suitable for optogenetics. By way of example, and not limitation the wavelength may include a blue light (˜470 nm). By way of example, and not limitation the wavelength may include yellow/green light (˜530-590 nm). By way of example, and not limitation the wavelength may include red light (˜620-700 nm).

The conduit 210 operatively couples an at least one optic cable 225. The at least one optic cable 225 is configured to receive the light 220 emitted from the at least one optical emitter 215. The at least one optic cable 225 may, for example, advantageously direct the light 220 emitted from the at least one optical emitter 215 to a target spot. The target spot may, for example, include genetically modified neurons configured to undergo optogenetics.

The MMFMM 200 includes two or more electrodes 230A and 230B, at least one pressure sensor 235, a valve 240, connectors 245, and an MMFMME 250. The two or more electrodes 230A and 230B, at least one pressure sensor 235, a valve 240, connectors 245, and an MMFMME 250 may, for example, be configured in a substantially similar arrangement to the two or more electrodes 140A and 140B, the at least one pressure sensor 145, the valve 150, the connectors 160, and the MMFMME 155. The MMFMM 200 may, for example, advantageously be employed in an ambulatory setting.

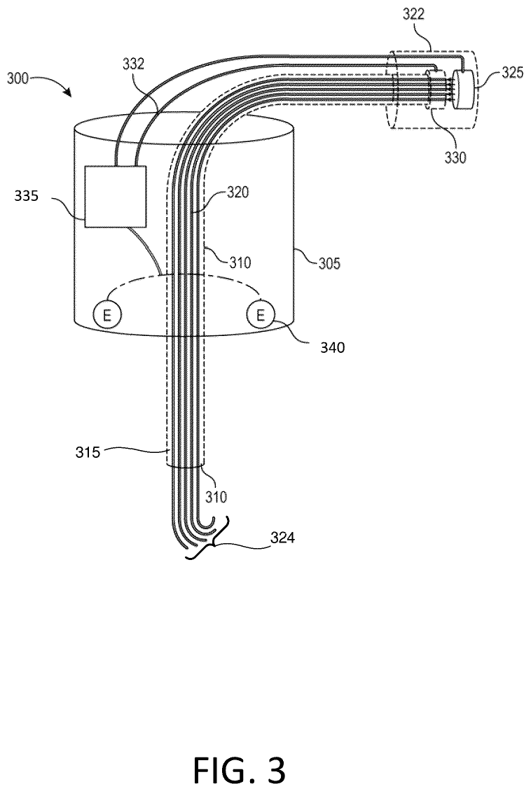

FIG. 3 depicts a schematic of another exemplary embodiment of an MMFMM 300. The MMFMM 300 includes a housing 305. The housing 305 encloses a conduit 310. The conduit 310 extends in a substantially similar arrangement to the conduit 125. The conduit 310 includes a lumen 315. Within the lumen 315, one or more optic cables 320 are enclosed. The proximal end of the conduit 310 and the proximal end of the one or more optic cables 320 are enclosed within an external housing 322. The external housing 322 may, for example, be positioned outside a patient's skull. The distal end of the one or more optic cables 320 extends out from the distal end of the conduit 310. The distal end of the one or more optic cables 320 includes one or more angled edges 324.

Enclosed within the external housing 322 is an at least one optical emitter 325. The at least one optical emitter 325 may, for example, emit light into the one or more optic cables 320. The one or more optic cables 320 may, for example, receive the light emitted from the at least one optical emitter 325 and emit said light out through the one or more angled edges 324. The one or more angled edges 324 may, for example, advantageously direct the light emitted from the one or more angled edges 324 at a particular angle to a target spot. The target spot may, for example, include genetically modified neurons configured to undergo optogenetics. Each of the one or more angled edges 324 may, for example, include a different angle at which it directs light.

The conduit 310 may, for example, operatively couple a cable deployment device 330. The cable deployment device 330 may, for example, be configured to rotate the conduit 310. The cable deployment device 330 may, for example, be configured to push or pull the conduit 310. The cable deployment device 330 is positioned at the proximal end of the conduit 310 wherein movement of the cable deployment device may, for example translate to movement through pull-wires (not depicted) embedded within the conduit 310 such that the pull wires adjust the curvature and direction of the conduit 310.

The cable deployment device 330 may, for example, be configured to independently and selectively rotate each of the one or more optic cables 320. The cable deployment device 330 may, for example, be configured to independently and selectively push or pull the one or more optic cables 320 further in or out of the conduit 310. For example, the proximal end of the one or more optic cables 320 may include a cable deployment device 330 wherein movement of the cable deployment device 330 translates to movement through pull-wires (not depicted) embedded within the one or more optic cables 320 such that the pull wires adjust the curvature and direction of the one or more optic cables 320. The cable deployment device 330 may, for example, advantageously enable the precise adjustment of the position of the one or more optic cables 320 such that the target spot of the angled edges 324 may be precisely adjusted.

The cable deployment device 330 may, for example, be manually operated. The cable deployment device 330 may, for example, be automatically operated. For example, the cable deployment device 330 and the at least one optical emitter 325 may communicably connect via connectors 332 to an MMFMME 335 such that the MMFMME 335 may automatically operate the cable deployment device 330 and the at least one optical emitter 325.

The MMFMM 300 includes two or more electrodes 340. The two or more electrodes 340 may, for example, be arranged in a substantially similar manner to the two or more electrodes 140A and 140B as well as the two or more electrodes 230A and 230B. The MMFMM 300 may, for example, include an at least one pressure sensor (not depicted) and valve (not depicted) arranged in a substantially similar manner to the at least one pressure sensor 145 and 235 and the valve 150 and 240. The MMFMM 300 may, for example, advantageously be employed in an outpatient setting.

FIG. 4 is a block diagram depicting an exemplary multi-modal fluid management module engine (MMFMME) 400. The MMFMME 400 includes a processor 405. The processor 405 is coupled to a signal storage memory 410. The signal storage memory 410 includes an electrode data store 415. The electrode data store 415 may, for example, store waveform EEG activity recorded by two or more electrodes, such as the embodiments described with reference to, for example, FIGS. 1-3 (electrodes 140A, 140B, 230A, 230B, and 340), communicably coupled to the MMFMME 400. The signal storage memory 410 includes a pressure sensor data store 420. The pressure sensor data store 420 may, for example, store waveform CSF pressure data recorded by at least one pressure sensor, such as the embodiments described with reference to, for example, FIGS. 1-2 (at least one pressure sensor 145 and 235) communicably coupled to the MMFMME 400. The signal storage memory 410 includes a brain activity data store 425. The brain activity data store 425 may, for example, store brain activity data recorded from an external medical device communicably coupled to an MMFMME. By way of example, and not limitation the external medical device may include an EEG machine. By way of example, and not limitation the external medical device may include a functional magnetic resonance imaging (fMRI) machine. By way of example, and not limitation the external medical device may include a magnetoencephalography (MEG) machine

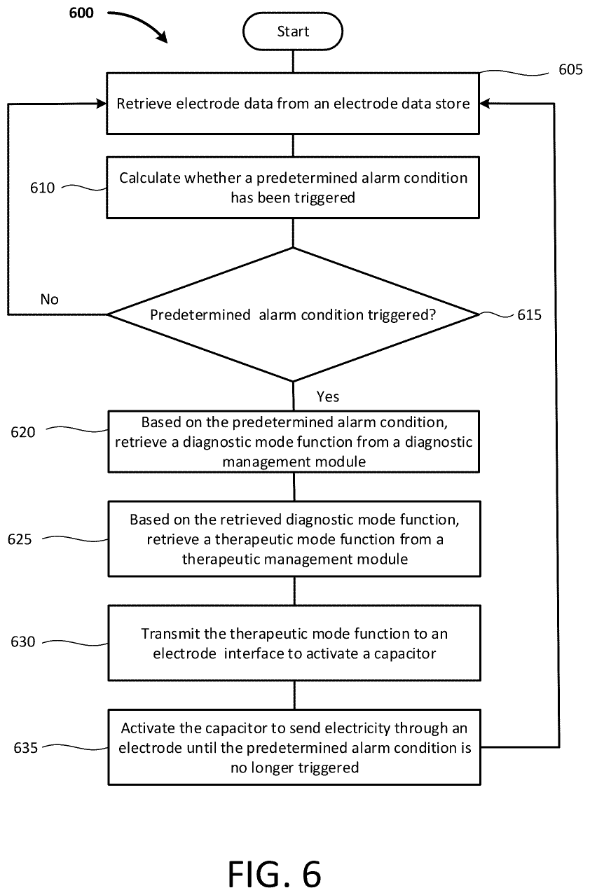

The processor 405 is coupled to a data store 430. The data store 430 includes a parameter data store 435. The parameter data store 435 includes mode shift values 440 and alarm condition threshold values 445. The parameter data store 435, mode shift values 440, and alarm condition threshold values 445 may, for example, be arranged and operate in a substantially manner to the parameter data store 565, mode shift values 565a, and alarm condition threshold values 565b described at least with reference to FIGS. 5-6 of U.S. application Ser. No. 19/053,113, titled “Multi-Modal Physiological Sensor,” filed by Samuel Robert Browd, et al., on Feb. 12, 2025.

The alarm condition threshold values 445 may, for example, further include brain activity threshold values, that when reached, cause the processor 405 to generate an alarm condition. Exemplary brain activity threshold values may include measured disruptions or deviations from the normal electrical signals and rhythms in the brain, which may manifest as seizures, changes in awareness, or other neurological symptoms.

The data store 430 includes a diagnostic management module 450. The diagnostic management module 450 includes diagnostic mode functions 450a. The diagnostic mode functions 450a may, for example, include diagnostic information corresponding to alarm condition threshold values 445. For example, alarm condition threshold values 445 may include an abnormal measurement of EEG activity revealed in a spike or sharp wave such that the corresponding diagnostic information may, for example, include a seizure.

The data store 430 includes a therapeutic management module 455. The therapeutic management module 455 includes therapeutic mode functions 455a. The therapeutic mode functions 455a may, for example, include therapeutic information corresponding to diagnostic mode functions 450a. For example, the diagnostic mode function 450a may, for example, include a seizure such that the corresponding therapeutic information includes producing electrical impulses in the section of the brain detected to be causing the seizure to stop the brain signals causing the seizure.

The processor 405 may, for example, transmit the therapeutic mode functions 455a to an electrode interface 460. The electrode interface 460 may, for example, operably couple two or more electrodes, such as the embodiments described with reference to, for example, FIGS. 1-3 (electrodes 140A, 140B, 230A, 230B, and 340), communicably coupled to the MMFMME 400. The electrode interface 460 may, for example, transmit the therapeutic mode functions 455a to a capacitor 465. The capacitor may, for example, electrically couple the two or more electrodes such that the capacitor is configured produce electrical impulses within the two or more electrodes. Additionally, the electrode interface may, for example, retrieve waveform EEG activity recorded by two or more electrodes and transmit the waveform EEG activity to the processor 405. The processor 405 may, for example, transmit the waveform EEG activity to the electrode data store 415 and/or the data store 430.

The processor 405 is coupled to a pressure sensor interface 470. The pressure sensor interface 470 may, for example, operably couple an at least one pressure sensor, such as the embodiments described with reference to, for example, FIGS. 1-2 (at least one pressure sensor 145 and 235), communicably coupled to the MMFMME 400. The pressure sensor interface 470 may, for example, retrieve waveform CSF pressure data recorded by the at least one pressure sensor and transmit the retrieved waveform CSF pressure data to the processor 405. The processor 405 may, for example, transmit the waveform CSF pressure data to the pressure sensor data store 420 and/or the data store 430. The CSF pressure data transmitted to the data store 430 may, for example, trigger alarm condition threshold values 445. For example, the alarm condition threshold values 445 may include a CSF pressure above 20 millimeters of mercury (mmHg). The alarm condition threshold values 445 may, for example, correspond to a diagnostic mode function 450a, for example, a CSF pressure above 20 millimeters of mercury (mmHg) may correspond to a diagnostic mode function 445a of intracranial hypertension. The diagnostic mode function 450a may, for example, correspond to a therapeutic mode function, for example, the diagnostic mode function 450a of intracranial hypertension may, for example, correspond to a therapeutic mode function to drain CSF from a CSF filled region of the brain.

The processor 405 may, for example, transmit the therapeutic mode function 455a to a valve interface 475. The valve interface 475 may, for example, operatively couple a valve, such as the embodiments described with reference to, for example, FIGS. 1-2 (valve 150 and 240). The valve interface 475 may, for example, receive a therapeutic mode function 455a such that the valve interface 475 configures the valve to increase the rate of CSF flow within a conduit in fluid communication with a CSF filled region of the brain when the pressure exceeds alarm condition threshold values 445 and decrease the rate of CSF flow when the pressure falls below alarm condition threshold values 445.

The processor 405 couples an optical emitter interface 480. The optical emitter interface 480 may, for example, operatively couple at least one optical emitter, such as the embodiments described with reference to, for example, FIGS. 1-3 (at least one optical emitter 135, 215, and 325), communicably coupled to the MMFMME 400. The processor may, for example, transmit a therapeutic mode function 455a to the optical emitter interface 480 such that optical emitter interface 480 configures the at least one optical emitter to emit light. The light may, for example, include light at a wavelength suitable for optogenetics. For example, the therapeutic mode function 455a transmitted to the optical emitter interface may include to either increase or dampen brain activity in a specific region of the brain configured to undergo optogenetics. The optical emitter interface 480 may, for example, direct the at least one optical emitter to emit light suitable for optogenetics at the specific brain region to either increase or dampen brain activity in that specific region of the brain. The optical emitter interface 480 may, for example, remotely control a cable deployment device, such as the embodiments described with reference to, for example, FIG. 3 (cable deployment device 330), such that the cable deployment device is configured to adjust the precise angle and location at which the at least one optical emitter emits light.

The processor 405 couples a communication interface 485. The communication interface 485 may, for example, communicably couple the MMFMME 400 to an external device 490. For example, the external device 490 may include a cloud server. The communication interface may, for example, transmit data stored in the signal storage memory 410 to a cloud interface. The external device 490 may, for example, include an app on an external device. The app may be configured to enable authorized users to access and monitor the status of the implanted device. In some implementations, the app on the external device may remotely control various functions of the MMFMME 400. The external device 490 may, for example, include a medical device. The medical device may, for example, include a medical device configured to record brain activity. By way of example, and not limitation the medical device may include an EEG machine. By way of example, and not limitation the medical device may include a fMRI machine. By way of example, and not limitation the medical device may include a MEG machine. The MMFMME 400 may, for example, include a power source 495.

In some embodiments, an MMFMM may, for example, perform operations substantially similar to the set of operations 600 as described at least with reference to FIG. 6 of U.S. application Ser. No. 19/053,113, titled “Multi-Modal Physiological Sensor,” filed by Samuel Robert Browd, et al., on Feb. 12, 2025.

FIG. 5 is a flowchart illustrating an exemplary method of regulating intracranial pressure including a set of exemplary operations 500 performed by a MMFMME, such as the embodiment described with reference to, for example, FIG. 4 (MMFMME 400). The set of operations 500 begins at a step 505, in which the processor retrieves pressure sensor data from a pressure sensor data store. The pressure sensor data may, for example, have been recorded by an at least one pressure sensor, such as the embodiments described with reference to, for example, FIGS. 1-2 (at least one pressure sensor 145 and 235), operatively coupled to the MMFMME via a pressure sensor interface.

At step 510 the processor calculates whether a predetermined alarm condition has been triggered based on the retrieved pressure sensor data. The predetermined alarm conditions may, for example, be stored in a parameter data store, such as the embodiment described with reference to, for example, FIG. 4 (parameter data store 435). A predetermined alarm condition may, for example, include a CSF pressure above 20 mmHg.

At a step 515, the processor reaches a decision point where the processor decides whether a predetermined alarm condition has been triggered based on the retrieved pressure sensor data. If the processor determines no, a predetermined alarm condition has not been triggered, the processor reverts to step 505. If the processor determines yes, a predetermined alarm condition has been triggered, the processor proceeds to a step 520.

At a step 520, based on the predetermined alarm condition, the processor retrieves a diagnostic mode function from a diagnostic management module. For example, a predetermined alarm condition including a CSF pressure above 20 mmHg may correspond to a diagnostic mode function of intracranial hypertension.

At a step 525, based on the retrieved diagnostic mode function, the processor retrieves a therapeutic mode function from a therapeutic management module. For example, a diagnostic mode function of intracranial hypertension may, for example, correspond to a therapeutic mode function of draining CSF from a ventricle in the brain.

At a step 530, the processor transmits the therapeutic mode function to a valve interface such that the valve interface activates a valve, such as the embodiments described with reference to, for example, FIGS. 1-2 (valve 150 and 240). For example, the processor may, for example, transmit the therapeutic mode function of draining CSF from a ventricle in the brain to the valve interface. The valve interface may, for example, operatively couple the valve operatively coupled to a conduit, such as the embodiments described with reference to, for example, FIGS. 1-3 (conduit 125, 210, and 310), in fluid communication with the ventricle in the brain such that the valve is configured to adjust the rate of CSF flow through the conduit. The therapeutic mode function of draining CSF from a ventricle in the brain transmitted to the valve interface may, for example, trigger the valve interface to signal to the valve to increase the CSF flow rate within the conduit. At a step 535, the valve drains CSF until the predetermined alarm condition is no longer triggered. For example, the processor may retrieve pressure sensor data from a pressure sensor data store that indicates the predetermined alarm condition is no longer triggered. The set of operations 500 then reverts to step 505 to continue to retrieve pressure sensor data from a pressure sensor data store.

FIG. 6 is a flowchart illustrating an exemplary method of modulating brain activity via electrodes including a set of exemplary operations 600 performed by a MMFMME such as the embodiment described with reference to, for example, FIG. 4 (MMFMME 400). The set of operations 600 begins at a step 605, in which a processor retrieves electrode data from an electrode data store. The electrode data may, for example, have been recorded by two or more electrodes, such as the embodiments described with reference to, for example, FIGS. 1-3 (electrodes 140A, 140B, 230A, 230B, and 340) operatively coupled to the MMFMME via an electrode interface.

At a step 610, the processor calculates whether a predetermined alarm condition has been triggered based on the retrieved electrode data. The predetermined alarm conditions may, for example, be stored in a parameter data store, such as the embodiment described with reference to, for example, FIG. 4 (parameter data store 435). A predetermined alarm condition may, for example, include waveform EEG activity indicating spikes and sharp waves in a specific area of the brain.

At a step 615, the processor reaches a decision point where the processor decides whether a predetermined alarm condition has been triggered based on the retrieved electrode data. If the processor determines no, a predetermined alarm condition has not been triggered, the processor reverts to step 605. If the processor determines yes, a predetermined alarm condition has been triggered, the processor proceeds to a step 620.

At a step 620, based on the predetermined alarm condition, the processor retrieves a diagnostic mode function from a diagnostic management module. For example, a predetermined alarm condition including waveform EEG activity indicating spikes and sharp waves in a specific area of the brain may, for example, correspond with a diagnostic mode function of a seizure.

At a step 625, based on the retrieved diagnostic mode function, the processor retrieves a therapeutic mode function from a therapeutic management module. For example, a diagnostic mode function of a seizure may, for example, correspond to a therapeutic mode function of producing electrical impulses in the specific area of the brain causing the seizure.

At a step 630, the processor transmits the therapeutic mode function to an electrode interface such that the electrode interface activates a capacitor. For example, the processor may, transmit the therapeutic mode function of producing electrical impulses in the specific area of the brain causing the seizure to the electrode interface. The electrode interface may, for example, operatively couple a capacitor operatively coupled to two or more electrodes in electrical contact with the specific area of the brain causing the seizure such that the capacitor is configured to produce electrical impulses in the two or more electrodes, such as the embodiments described with reference to, for example, FIGS. 1-3 (electrodes 140A, 140B, 230A, 230B, and 340). The therapeutic mode function of producing electrical impulses in the specific area of the brain causing the seizure transmitted to the electrode interface may, for example, trigger the electrode interface to signal to the capacitor to produce electrical impulses in the two or more electrodes.

At a step 635, the two or more electrodes produce electrical impulses in the specific area of the brain causing the seizure until the predetermined alarm condition is no longer triggered. For example, the processor may retrieve electrode data from an electrode data store that indicates the predetermined alarm condition is no longer triggered. The set of operations 600 then reverts to step 605 to continue to retrieve electrode data from an electrode data store.

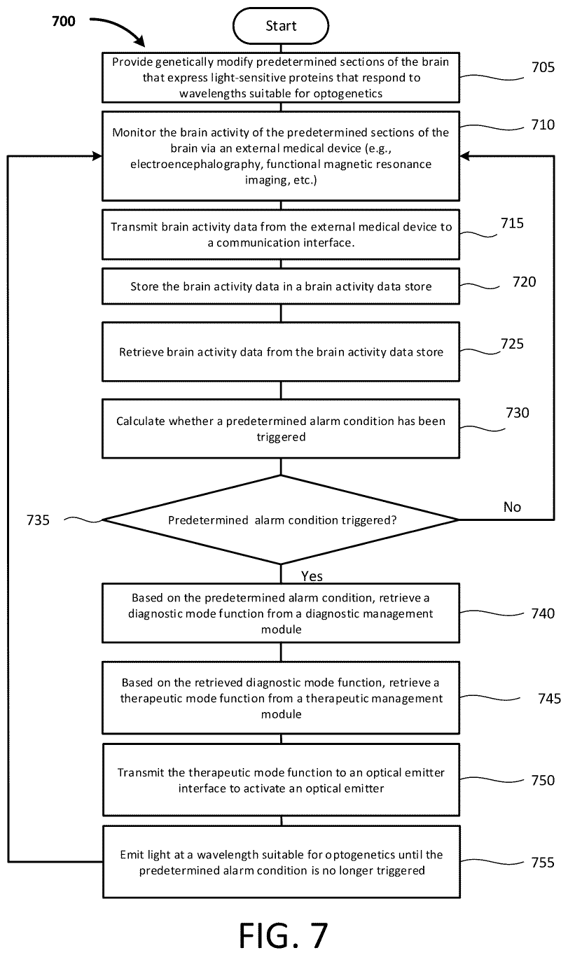

FIG. 7 is a flowchart illustrating an exemplary method 700 of modulating brain activity via optogenetics including a set of exemplary operations performed by a MMFMME, such as the embodiment described with reference to, for example, FIG. 4 (MMFMME 400). The method 700 begins at a step 705, in which genetically modified predetermined sections of a patient's brain that express light-sensitive proteins that respond to wavelengths suitable to optogenetics are provided. The predetermined section of the patient's brain may, for example, include the subgenual anterior cingulate cortex (sgACC).

At a step 710, an external medical device communicably coupled to the MMFMME via a communication interface, monitors the brain activity of the genetically modified predetermined sections of a patient's brain. By way of example, and not limitation the external medical device may include an EEG machine. By way of example, and not limitation the external medical device may include a fMRI machine. By way of example, and not limitation the external medical device may include a MEG machine.

At a step 715, the external medical device transmits the recorded brain activity data to the communication interface. At a step 720, the processor retrieves the recorded brain activity data from the communication interface and stores the recorded brain activity data in a brain activity data store.

At a step 725, the processor retrieves the brain activity data from the brain activity data store. At a step 730, the processor calculates whether a predetermined alarm condition has been triggered based on the retrieved electrode data. The predetermined alarm conditions may, for example, be stored in a parameter data store, such as the embodiment described with reference to, for example, FIG. 4 (parameter data store 435). A predetermined alarm condition may, for example, include increased brain activity in the sgACC.

At a step 735, the processor reaches a decision point where the processor decides whether a predetermined alarm condition has been triggered based on the retrieved brain activity data. If the processor determines no, a predetermined alarm condition has not been triggered, the processor reverts to step 710. If the processor determines yes, a predetermined alarm condition has been triggered, the processor proceeds to a step 740.

At a step 740, based on the predetermined alarm condition, the processor retrieves a diagnostic mode function from a diagnostic management module. For example, a predetermined alarm condition including increased brain activity in the sgACC may, for example, correspond with a diagnostic mode function of Major Depressive Disorder (MDD).

At a step 745, based on the retrieved diagnostic mode function, the processor retrieves a therapeutic mode function from a therapeutic management module. For example, a diagnostic mode function of MDD may, for example, correspond to a therapeutic mode function of utilizing optogenetic inhibition to reduce hyperactivity in the sgACC.

At a step 750, the processor transmits the therapeutic mode function to an optical emitter interface such that the optical emitter interface activates an at least one optical emitter, such as the embodiments described with reference to, for example, FIGS. 1-3 (at least one optical emitter 135, 215, and 325). For example, the processor may, for example, transmit the therapeutic mode function of utilizing optogenetic inhibition to reduce hyperactivity in the sgACC to the optical emitter interface. The optical emitter interface may, for example, operatively couple an at least one optical emitter configured to direct light at wavelength suitable for optogenetics to the sgACC. The therapeutic mode function of utilizing optogenetic inhibition to reduce hyperactivity in the sgACC transmitted to the optical emitter interface may, for example, trigger the optical emitter interface to signal to the at least one optical emitter to direct light at wavelength suitable for optogenetics to the sgACC.

At a step 755, the optical emitter emits light at a wavelength suitable for optogenetics until the predetermined alarm condition is no longer triggered. For example, the processor may retrieve brain activity data from a brain activity data store that indicates the predetermined alarm condition is no longer triggered. The method 700 then reverts to step 710 to continue to monitor the brain activity of the predetermined sections of the patient's brain via the external medical device.

In some embodiments, a conduit, such as the embodiments described with reference to, for example, FIGS. 1-3 (conduit 125, conduit 210, conduit 310) may, for example, provide a catheter. In some implementations, the conduit may, for example, provide an external ventricular drain (EVD). In various embodiments, apparatus and methods may involve preventing occlusion within the conduit. For example, the operations of an MMFMME, such as the embodiments described with reference to, for example, FIGS. 1-4 (MMFMME 155, MMFMME 250, MMFMME 335, MMFMME 400), may include retrieving a mode shift function such that an MMFMM, such as the embodiments described with reference to, for example, FIGS. 1-3 (MMFMM 100, MMFMM 200, MMFMM 300), may prevent occlusion of the conduit via an electrical charge from the two or more electrodes, for example, the embodiments described with reference to, for example, FIGS. 1-3 (electrodes 140A, 140B, 230A, 230B, and 340). The operations of the MMFMME may, for example, include retrieving a mode shift function such that the MMFMM may prevent occlusion of the conduit through flushing back CSF into the conduit via a valve, for example, the valve described with reference to, for example, FIGS. 1-2 (valve 150, valve 240).

In some embodiments, an MMFMM, such as the embodiments described with reference to, for example, FIGS. 1-3 (MMFMM 100, MMFMM 200, MMFMM 300) may, for example, be configured to measure brain compliance (e.g., the brain's ability to accommodate changes in pressure). For example, through flushing back CSF into a conduit, such as the conduits described with reference to, for example, FIGS. 1-3 (conduit 125, conduit 210, conduit 310), via a valve, such as the embodiments described with reference to, for example, FIGS. 1-2 (valve 150, valve 240), the flushed backed CSF may cause the brain to recoil and generate a pulse waveform, providing a measure of the brain's compliance.

In some implementations, two or more electrodes, such as the embodiments described with reference to, for example, FIGS. 1-3 (electrodes 140A, 140B, 230A, 230B, and 340), may, for example, be a metal wire, a carbon nanotube, or a conductive polymer. The two or more electrodes may, for example, be controlled by an MMFMME, for example, such as the embodiments described with reference to, for example, FIGS. 1-4 (MMFMME 155, MMFMME 250, MMFMME 335, MMFMME 400), to adjust the amplitude, duration, frequency, or waveform of the electrical stimulation. In some implementations, the MMFMME may, for example, independently modulate either or both currents in either or both of the two or more electrodes.

In accordance with some embodiments, an exemplary MMFMM, such as those described with reference to, for example, FIGS. 1-3 (MMFMM 100, MMFMM 200, MMFMM 300), may be configured to detect the activity or sleep state of the patient. For example, the MMFMM may be configured to detect the activity or sleep state based on one or more signals from the patient's brain detected by the MMFMM, for example, through EEG monitoring of the patient's brain. The MMFMM may, for example, utilize electrode stimulation to modulate sleep.

In accordance with some exemplary embodiments, an MMFMM, for example, the MMFMM described with reference to FIGS. 1-3 (MMFMM 100, MMFMM 200, MMFMM 300) may, for example, operatively couple other implanted devices in another location in the brain. For example, an MMFMM implanted in the brain may operatively couple another MMFMM implanted in another location in the brain.

Although various embodiments have been described with reference to the figures, other embodiments are possible.

Although an exemplary system has been described with reference to FIGS. 1-7, other implementations may be deployed in other industrial, scientific, medical, commercial, and/or residential applications. For example, the system could be employed in the medical and healthcare industry for patients with hydrocephalus, traumatic brain injury, or other neurological diseases. Additionally, integrated optogenetic stimulation and electrode-based neuromodulation may enable precise control over neural circuits, offering potential therapeutic interventions, for example, in diseases like epilepsy, Parkinson's disease, or treatment-resistant depression. The system may, for example, be employed with brain-computer interface (BCI). The system may, for example, be employed in the pharmaceutical and research industry for real-time monitoring of the effects of treatments on the brain. The system may, for example, be deployed in the artificial intelligence (AI) and data science industry. For example, the collection of robust datasets of brain activity and CSF pressure signals may be utilized to enhance AI models that interpret brain signals and improve machine learning algorithms that predict neurological conditions.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a 9V (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

1-19. (canceled)

20. A multi-modal fluid management module system (MMFMMS) comprising:
a housing configured to operatively contact a dura while implanted through an aperture in a patient's skull;
a conduit configure to extend into the housing and comprising a proximal aperture end in fluid communication with a distal aperture end, the conduit configured to operatively contact a cerebrospinal fluid (CSF) filled region of a brain via an aperture in the dura;
a processor disposed in the housing;
two or more electrodes configured to operatively couple to the processor and configured to be in electrical contact with a first selected region within a skull cavity in a first mode;
an at least one optical emitter configured to operatively couple to the processor and configured to emit light within the brain at a wavelength suitable for optogenetics in a second mode;
an at least one pressure sensor configured to operatively couple to the processor and configured to mechanically couple to the conduit in a third mode;
a valve configured to operatively couple to the processor and configured to operatively couple to the conduit, the valve configured to control the rate of fluid flow in and out of the conduit in a fourth mode; and
a data store operatively coupled to the processor and containing instructions that, when operated by the processor, cause the processor to perform operations to selectively switch between the first mode to monitor activity of the brain and the second mode to modulate activity of the brain, the operations comprising:
retrieve, from the data store, a mode shift function indicative of a state of an alarm condition; and,
transition from the first mode to the second mode if the retrieved mode shift function indicates the retrieved alarm condition is triggered.

21. The MMFMMS of claim 20, wherein the at least one optical emitter helically wraps along the outer surface of the conduit.

22. The MMFMMS of claim 20, wherein the at least one optical emitter further comprises at least one optic cable operatively coupled to the conduit, wherein the at least one optic cable is configured to direct light emitted from the optical emitter to corresponding independent targets within a brain.

23. The MMFMMS of claim 20, wherein the at least one optical emitter further comprises at least one optic cable configured to receive light emitted from the optical emitter and positioned within a lumen of the conduit wherein a distal end of the at least one optical cable extends out of the distal aperture end of the conduit, the distal end of the at least one optic cable comprising independently selected distal angles configured to be independently rotatably controlled and axially positionable to direct light emitted from the optical emitter to corresponding independent targets within a brain.

24. The MMFMMS of claim 20, wherein the at least one optical emitter further comprises at least one optic cable configured to receive light emitted from the optical emitter and positioned within a lumen of the conduit wherein a distal end of the at least one optical cable extends out of the distal aperture end of the conduit, the distal end of the at least one optic cable comprising independently selected distal angles wherein the conduit further comprises pull-wires embedded within the conduit and a cable deployment device operatively coupled to the conduit and positioned at the proximal end of the conduit wherein movement of the cable deployment device is configured to translate to movement of the pull-wires embedded within the conduit such that the pull wires are configured to adjust the curvature and direction of the conduit.

25. The MMFMMS of claim 20, wherein the two or more electrodes helically wrap along the outer surface of the conduit.

26. The MMFMMS of claim 20, wherein the two or more electrodes are communicably coupled to an electrode interface, wherein the electrode interface is positioned within the housing and electrically coupled to a capacitor positioned within the housing, such that the electrode interface is configured to provide an electrical charge to the two or more electrodes via the capacitor.

27. The MMFMMS of claim 20, wherein the data store operatively coupled to the processor and contains further instructions that, when operated by the processor, cause the processor to perform operations to selectively switch between the third mode to monitor the CSF pressure of the brain, and the fourth mode to modulate CSF pressure of the brain, the operations comprising: retrieve, from the data store, a mode shift function indicative of a state of an alarm condition; and, transition from the third mode to the fourth mode if the retrieved mode shift function indicates the retrieved alarm condition is triggered.

28. The MMFMMS of claim 20, wherein the housing further encloses a communication module communicably coupled to the processor and communicably coupled to an external interface such that the MMFMMS is configured to be remotely monitored and controlled via the external interface.

29. A multi-modal fluid management module system (MMFMMS) comprising:
a housing configured to operatively contact a dura while implanted through an aperture in a patient's skull;
a conduit configured to extend into the housing and comprising a proximal aperture end in fluid communication with a distal aperture end, the conduit configured to operatively contact a cerebrospinal fluid (CSF) filled region of a brain via an aperture in the dura;
a processor disposed in the housing;
two or more electrodes configured to operatively couple to the processor and configured to be in electrical contact with a first selected region within a skull cavity in a first mode;
an at least one optical emitter configured to operatively couple to the processor and configured to emit light within a brain at a wavelength suitable for optogenetics in a second mode; and,
a data store operatively coupled to the processor and containing instructions that, when operated by the processor, cause the processor to perform operations to selectively switch between the first mode to monitor activity of the brain and the second mode to modulate activity of the brain, the operations comprising:
retrieve, from the data store, a mode shift function indicative of a state of an alarm condition;
transition from the first mode to the second mode if the retrieved mode shift function indicates the retrieved alarm condition is triggered.

30. The MMFMMS of claim 29, wherein the at least one optical emitter helically wraps along the outer surface of the conduit.

31. The MMFMMS of claim 29, wherein the at least one optical emitter further comprises at least one optic cable operatively coupled to the conduit, wherein the at least one optic cable is configured to direct light emitted from the at least one optical emitter to corresponding independent targets within a brain.

32. The MMFMMS of claim 29, wherein the at least one optical emitter further comprises at least one optic cable configured to receive light emitted from the optical emitter and positioned within a lumen of the conduit wherein a distal end of the at least one optical cable extends out of the distal aperture end of the conduit, the distal end of the at least one optic cable comprising independently selected distal angles configured to be independently rotatably controlled and axially positionable to direct light emitted from the optical emitter to corresponding independent targets within a brain.

33. The MMFMMS of claim 29, wherein the at least one optical emitter further comprises at least one optic cable configured to receive light emitted from the optical emitter and positioned within a lumen of the conduit wherein a distal end of the at least one optical cable extends out of the distal aperture end of the conduit, the distal end of the at least one optic cable comprising independently selected distal angles wherein the conduit further comprises pull-wires embedded within the conduit and a cable deployment device operatively coupled to the conduit and positioned at the proximal end of the conduit wherein movement of the cable deployment device is configured to translate to movement of the pull-wires embedded within the conduit such that the pull wires are configured to adjust the curvature and direction of the conduit.

34. The MMFMMS of claim 29, wherein the two or more electrodes helically wrap along the outer surface of the conduit.

35. The MMFMMS of claim 29, wherein the two or more electrodes are communicably coupled to an electrode interface, wherein the electrode interface is positioned within the housing and electrically coupled to a capacitor positioned within the housing, such that the electrode interface is configured to provide an electrical charge to the two or more electrodes via the capacitor.

36. The MMFMMS of claim 29, wherein the first selected region within a skull cavity comprises a region of the brain.

37. The MMFMMS of claim 29, wherein the first selected region within a skull cavity comprises a region of the dura.

38. The MMFMMS of claim 29, wherein the conduit provides a fluid communication path that extends through the housing and radially from the housing into a subcutaneous path to a location within a patient's body for drainage.

39. The MMFMMS of claim 29, wherein the housing further encloses a communication module communicably coupled to the processor and communicably coupled to an external interface such that the MMFMMS is configured to be remotely monitored and controlled via the external interface.
